SEQUENCE LISTING (3 sequences)

SEQ ID NO: 1 - protein, 15 aa, Artificial sequence
  epitope peptide sequence of collagen type VI alpha 1
ADWGQSRDAEEAISQ

SEQ ID NO: 2 - protein, 15 aa, Artificial sequence
  Modified N-terminal epitope sequence as a screening peptide
ADWGQSRDAEEAISQ

SEQ ID NO: 3 - protein, 18 aa, Artificial sequence
  Modified N-terminal epitope sequence as an immunogenic peptide
ADWGQSRDAEEAISQGGC

(19) United States
(12) Patent Application Publication
(10) Pub. No.: US 2016/0077108 A1
(43) Pub. Date: Mar. 17, 2016

(54) Collagen Type VI Alpha-1 Assay

(71) Applicant: NORDIC BIOSCIENCE A/S, Herlev (DK)

(72) Inventors: Diana Julie Leeming, Espergaerde (DK); Anders Nedergaard, Kobenhavn N (DK); Morten Karsdal, Kobenhavn O (DK)

(73) Assignee: NORDIC BIOSCIENCE A/S, Herlev (DK)

(21) Appl. No.: 14/786,301

(22) PCT Filed: Apr. 23, 2014
(86) PCT No.: PCT/EP2014/058192

§ 371 Date: Oct. 22, 2015

(30) Foreign Application Priority Data
Apr. 24, 2013 (GB) 1307387.9

(51) Int. Cl. G01N 33/68; C07K 16/18

(52) U.S. Cl. CPC G01N 33/6887; C07K 16/18; G01N 2333/78; C07K 2317/34

(57) ABSTRACT

Provided is an antibody which binds to an epitope of collagen type VI alpha 1 comprised in the N-terminal globular domain internal amino acid sequence -ADWGQSRDAEEAISQ- (SEQ ID NO: 1). Also provided is a method of immunoassay for detecting in a biological sample an epitope comprised in the N-terminal globular domain internal amino acid sequence -ADWGQSRDAEEAISQ- (SEQ ID NO: 1) of collagen type VI alpha 1, by contacting the sample with the antibody, and determining the amount of binding of the antibody.

### TECHNICAL FIELD

The present invention relates to an antibody which binds to an epitope of collagen type VI alpha 1, and its use in a method of immunoassay for detecting and quantifying collagen type VI alpha 1.

### BACKGROUND ART

The extracellular matrix (ECM) is a supramolecular structure with the ability to form aggregates of proteins, thus forming a dynamic scaffold linking cells together in a three dimensional network. This scaffold controls cell-matrix interactions and cell fate through up and down regulation of proteases [1]. The ECM consists of collagens, laminins, proteoglycans, and other glycoproteins in various amounts and combinations, thereby providing a variety of biological components which can be modified by proteases to produce scaffolds with specific functions to meet the needs of the individual tissue [2].

### Collagens

The most abundant proteins in the extracellular matrix are members of the collagen family. Collagens are a group of proteins with characteristic fibrillar structures contributing to the extracellular scaffolding and are the major structural element of all connective tissues.

Collagens are also found in the interstitial tissue of virtually all parenchymal organs, where they contribute to the stability of tissues and organs and maintain their structural integrity. All members of the collagen family contain domains comprising repetitions of the proline rich tripeptide Gly-X-Y involved in the formation of trimeric collagen triple helices.

Knowledge of the molecular structure, biosynthesis, assembly and turnover of collagens is important in understanding pathological processes linked to many human diseases. The exploration of expression and function of the different collagen types also contributes to a better understanding of diseases which are based on molecular defects of collagen genes.

### Type VI Collagen

Type VI collagen is a heterotrimer of three different alpha-chains (alpha-1, alpha-2, alpha-3) with short triple helical domains and extended globular termini [3][4]. Following secretion into the extracellular matrix, type VI collagen aggregates into filaments and forms an independent microfibrillar network in virtually all connective tissues, except bone [5][6][7]. Type VI collagen fibrils appear on the ultrastructural level as fine filaments, microfibrils or segments with faint crossbanding of 110nm periodicity [7]-[12]. Defects in the corresponding type VI collagen genes have thus far only been associated with a range of congenital muscle dystrophy phenotypes, most notably but not limited to, the Bethlem and Ullrich Myopathies, which indicates an indispensable role in muscle tissue [13].

A method which accurately quantifies the amount of collagen type VI in a biological sample may allow a better understanding of collagen type VI pathologies or physiological processes affecting collagen type VI turnover such as muscle mass or muscle tissue turnover. Thus, there is an evident need for such a method.

WO 2013/011056 discloses methods of detecting and quantifying neo-epitopes of collagens including type VI in biofluid and using this to evaluate pathological conditions.

WO 2010/115749 discloses methods of diagnosis and quantitation of fibrosis comprising conducting an immunoassay to measure neo-epitope containing protein fragments naturally present in a biofluid sample, wherein said fragments may derive from collagen type VI, and associating an elevation of said measure in said patient above a normal level with the presence or extent of fibrosis.

EP 2051073 discloses methods of immunoassay and kits useful in the prediction of preterm cervical softening, said methods and kits comprising monoclonal antibodies which can recognise, amongst other things, collagen type VI.

### DESCRIPTION OF THE INVENTION

The herein described invention relates to an antibody directed to an epitope of collagen type VI alpha 1 in the N-terminal globular domain internal amino acid sequence -ADWGQSRDAEEAISQ- and a method of immunoassay for detecting and quantifying the amount of intact collagen type VI alpha 1 and fragments thereof comprising the amino acid sequence -ADWGQSRDAEEAISQ- (SEQ ID NO: 1).

None of the above cited art discloses the specific amino acid sequence ADWGQSRDAEEAISQ (SEQ ID NO: 1) or subsequences thereof or antibodies specific for said sequence and/or subsequences thereof.

In a first aspect, the present invention relates to an antibody, wherein said antibody binds to an epitope of collagen type VI alpha 1, said epitope being comprised in the N-terminal globular domain internal amino acid sequence -ADWGQSRDAEEAISQ- (SEQ ID NO: 1).

In a preferred embodiment of the invention, said antibody specifically reacts with an epitope of collagen type VI alpha 1, said epitope being comprised in the N-terminal globular domain internal amino acid sequence -ADWGQSRDAEEAISQ- (SEQ ID NO: 1).

In a preferred embodiment of the invention, said antibody is a monoclonal antibody, or a polyclonal antibody, or an antibody fragment.

In a preferred embodiment of the invention, said antibody binds to an epitope comprised in the N-terminal globular domain internal amino acid sequence -ADWGQSRDAEEAISQ- (SEQ ID NO: 1) of human or rodent collagen type VI alpha 1.

In another aspect, the present invention relates to a method of immunoassay for detecting in a biological sample an epitope comprised in the N-terminal globular domain internal amino acid sequence -ADWGQSRDAEEAISQ- (SEQ ID NO: 1) of collagen type VI alpha 1, said method comprising contacting said biological sample comprising said epitope comprised in said N-terminal globular domain internal amino acid sequence -ADWGQSRDAEEAISQ- (SEQ ID NO: 1) of collagen type VI alpha 1 with an antibody of the invention, and determining the amount of binding of said antibody.

In a preferred embodiment of the invention, said method of immunoassay is used to quantify the amount of intact collagen type VI alpha 1 and fragments thereof comprising the amino acid sequence ADWGQSRDAEEAISQ (SEQ ID NO: 1) in biofluid, wherein said biofluid may be, but is not limited to, serum or plasma.

In a preferred embodiment of the invention, said method of immunoassay may be, but is not limited to, a competition assay or a sandwich assay.

In a preferred embodiment of the invention, said method of immunoassay may be, but is not limited to, a radioimmunoassay or an enzyme-linked immunosorbent assay.

In a preferred embodiment of the invention, said method of immunoassay further comprises correlating the quantity of intact collagen type VI alpha 1 and fragments thereof comprising said amino acid sequence ADWGQSRDAEEAISQ (SEQ ID NO: 1) determined by said method with standard disease samples of known disease severity to evaluate the severity of a disease associated with collagen type VI alpha 1.

In a preferred embodiment of the invention, said method of immunoassay further comprises correlating the quantity of intact collagen type VI alpha 1 and fragments thereof comprising said amino acid sequence ADWGQSRDAEEAISQ (SEQ ID NO: 1) determined by said method with MRI-determined muscle volume.

In another aspect, the present invention relates to an assay kit for determining the quantity of intact collagen type VI alpha 1 and fragments thereof comprising the amino acid sequence ADWGQSRDAEEAISQ (SEQ ID NO: 1) in a biological sample, said kit comprising an antibody as described herein and at least one of:
a streptavidin coated 96 well plate
a biotinylated peptide Biotin-L-ADWGQSRDAEEAISQ (SEQ ID NO: 2), wherein L is an optional linker
a secondary antibody for use in a sandwich immunoassay
a calibrator peptide comprising the sequence ADWGQSRDAEEAISQ (SEQ ID NO: 1) or a portion thereof which binds to said antibody
an antibody biotinylation kit
an antibody HRP labeling kit
an antibody radiolabeling kit
an assay visualization kit

### DEFINITIONS

“IC6” is used to distinguish the herein described collagen type VI alpha 1 assay from collagen type VI alpha 1 assays known in the art.

“Antibody” as used herein refers to a monoclonal antibody, a polyclonal antibody, or an antibody fragment, such as Fab, F(ab′)₂, Fv, or scFv fragments etc., or a chemically modified derivative of any of these.

### FIGURES

FIG. 1: Calibration curve and inhibition of the competitive ELISA using human serum, human plasma and rat serum.

FIG. 2: The IC6 ELISA calibration curve plotted using a 4-parametric mathematical fit (y=(A-D)/(1+(x/ĈB)+D)) model.

FIG. 3: Collagen type VI alpha 1 as determined by the herein described IC6 ELISA plotted against muscle volume determined by MRI.

### EXAMPLES

### Materials and General Considerations

All reagents used in the experiments were high-standard chemicals from companies such as Merck (Whitehouse Station, N.J., USA) and Sigma Aldrich (St. Louis, Mo., USA). The synthetic peptides used for monoclonal antibody production and validation were 1) Immunogenic peptide: ADWGQSRDAEEAISQ-GGC-KLH (Keyhole Limpet Hemocyanin)(SEQ ID NO: 3) 2) Screening peptide: Biotin-ADWGQSRDAEEAISQ (SEQ ID NO: 2), and 3) Selection peptide: ADWGQSRDAEEAISQ (SEQ ID NO: 1). All synthetic peptides were purchased from the Chinese Peptide Company, Beijing, China.

### Example 1

### Monoclonal Antibody Generation

The antigen peptide amino acid sequence was generated from the consensus collagen type VI alpha 1 sequence (NP 001839). It was directed against the N-terminal globular domain and selected for minimization of homology to other human proteins and optimization of immunogenicity. The resulting epitope amino acid sequence was ADWGQSRDAEEAISQ (SEQ ID NO: 1). Generation of monoclonal antibodies was initiated by subcutaneous immunization of 4-5 week old Balb/C mice with 200 μl emulsified antigen and 50 μg of the collagen type VI alpha 1 epitope sequence conjugated to keyhole limpet hemocyanin ADWGQSRDAEEAISQ-GGC-KLH (SEQ ID NOL 3). The immunizations were repeated every 2 weeks until stable serum titer levels were reached. Blood samples were collected from the 2nd immunization. At each blood sampling, the serum titer was determined and the mouse with highest antiserum titer was selected for fusion. After the 4th immunization, this mouse was rested for 1 month and then boosted intravenously with 50 pg CO6-MMP (described in [14]) in 100 μl 0.9% sodium chloride solution three days before isolation of the spleen for cell fusion. The fusion procedure was performed as described by Gefter et al [15]. Briefly, mouse spleen cells were fused with SP2/0 myeloma fusion partner cells. The hybridoma cells were cloned using a semi-solid medium method and transferred into 96-well microtiter plates for further growth and incubated in a CO2-incubater. Standard limited dilution was used to promote monoclonal growth. The supernatants were screened for reactivity against the calibrator peptide and native material in an indirect ELISA using streptavidin-coated plates. Biotin-ADWGQSRDAEEAISQ (SEQ ID NO: 2) was used as screening peptide, while the free peptide ADWGQSRDAEEAISQ (SEQ ID NO: 1) was used as calibrator to test for further specificity of clones.

### Clone Characterisation

Native reactivity and affinity of the peptide were assessed using different biological materials such as human serum, human plasma and rat serum in a preliminary ELISA using the biotinylated peptide coater on a streptavidin coated microtitre plate and the supernatant from the growing monoclonal hybridoma cells. Specificity of the clones to a free peptide (ADWGQSRDAEEAISQ (SEQ ID NO: 1)) was tested. Isotyping of the monoclonal antibodies was performed using the Clonotyping System-HRP kit, cat. no. 5300-05 (Southern Biotech, Birmingham, Ala., USA). The selected clones were purified using protein G columns according to manufacturer's instructions (GE Healthcare Life Science, Little Chalfont, Buckinghamshire, UK). Selected monoclonal antibodies were labeled with horseradish peroxidase (HRP) using the Lightning link HRP labeling kit according to the instructions of the manufacturer (Innovabioscience, Babraham, Cambridge, UK).

### Clone selection

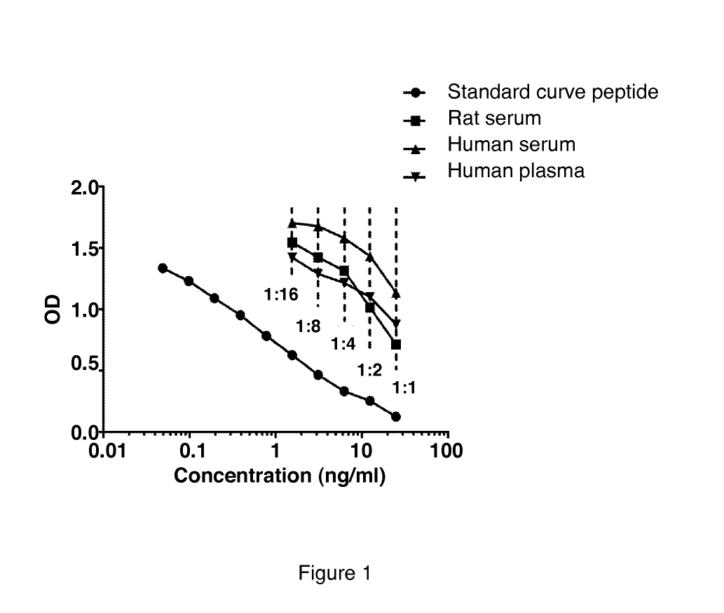

The subtype was determined to be an IgG1 subtype. Native reactivity was seen towards human serum, human plasma and rat serum (FIG. 1). The signal of the competitive ELISA was inhibited using dilutions from 1:2 to 1:16. Dilution of the native material approximately followed the same dilution pattern as the calibrator curve.

### Example 2

### IC6 Enzyme-Linked Immunosorbent Assay (ELISA)

### ELISA Assay Generation/Optimization

The buffer type, coater concentration, antibody concentration and incubation conditions were optimised using standard methods.

### IC6 ELISA Protocol

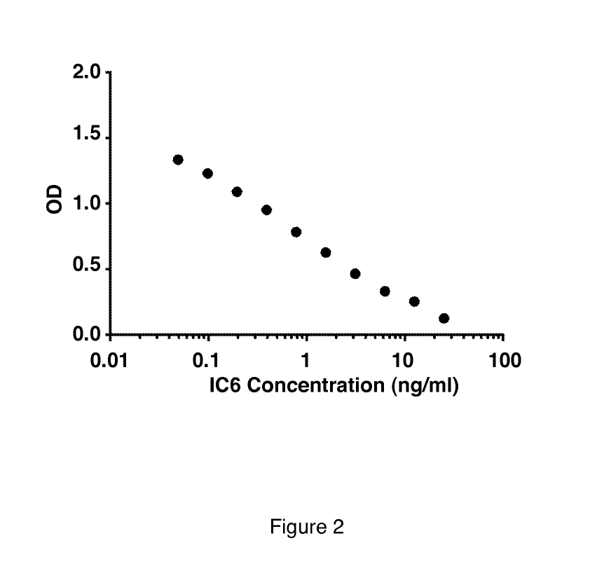

The competitive IC6 ELISA procedure was as follows: A 96-well streptavidin-coated ELISA plate from Roche, cat.11940279, was coated with the biotinylated peptide Biotin-ADWGQSRDAEEIAISQ (SEQ ID NOL 2) dissolved in coater buffer (25 mM PBS-BTB, pH 7.4) at 6 ng/ml in 100 μl, incubated for 30 min at 20° C. in the dark and subsequently washed in washing buffer (20 mM Tris, 50 mM NaCl, pH 7.2). Thereafter 20 μl of peptide calibrator or sample was added to the appropriate wells, followed by 100 pl of HRP-conjugated monoclonal antibody (described in example 1) dissolved in incubation buffer (25 mM PBS-BTB, pH 7.4) at 190 ng/ml. The plate was incubated for 20 hours at 4° C. and washed. Finally, 100 μl tetramethylbenzinidine (TMB) (Kem-En-Tec cat.: 4380H) was added, the plate was incubated for 15 min at 20° C. in the dark and the reaction was stopped by addition of 100 μl of stopping solution (1% H₂50₄). The plate was analyzed in an ELISA reader at 450 nm with 650 nm as the reference (Molecular Devices, SpectraMax M, Calif., USA). A calibration curve was plotted using a 4-parametric mathematical fit (y=(A−D)/(1+(x/ĈB)+D)) model (FIG. 2).

### Technical Evaluation of IC6 ELISA

From 2-fold dilutions of pooled serum and plasma samples, linearity was calculated as a percentage of recovery of the 100% sample. Dilution recovery was within 100 +/−20% (Table 1) for dilution ranges 1:2-1:8 for rat samples and through the full range (down to 1:16) for human samples.

TABLE 1
IC6 Assay dilution recovery. Dilution recovery is defined
as the signal multiplied by the dilution degree, relative
to the signal in the reference (undiluted) sample.

| | Rat | Human | EDTA- | Citrate- | |
|---|---|---|---|---|---|
| | serum | Serum | plasma | plasma | Heparin- |
| | (n = 4) | (n = 4) | (n = 2) | (n = 2) | plasma |
| Dilution factor | dRE % | dRE % | dRE % | sRE % | (n = 2) |
| Neat | 100 | 100 | 100 | 100 | 100 |
| 2x | 77 | 90 | 81 | 84 | 88 |
| 4x | 90 | 89 | 80 | 81 | 78 |
| 8x | 103 | 103 | 82 | 87 | 86 |
| 16x | 178 | 119 | 92 | 95 | 99 |

The lower detection limit (LDL) was calculated from 21 determinations of the lowest standard (the zero standard) and calculated as the mean +3× standard deviation. The lower LDL for the assay was 0.032 ng/mL. The inter- and intra-assay variation was determined by 10 independent runs of 5 QC samples, with each run consisting of two replicas of double determinations of the samples (Table 2).

TABLE 2
Intra- and inter-assay variability measured using 10 repetitive
measurements of the same human control samples on the same
plates or in individual plates, respectively.

| | | Intra-assay | Inter-assay |
|---|---|---|---|
| | Conc | variability | variability |
| Sample | (ng/ml) | (%) | (%) |
| HS1 | 0.76 | 15.34 | 14.80 |
| HS2 | 1.83 | 14.30 | 10.15 |
| HS3 | 1.94 | 10.71 | 8.68 |
| HS4 | 2.48 | 9.56 | 8.86 |
| HS5 | 2.40 | 11.20 | 11.18 |
| HS6 | 2.64 | 10.23 | 12.33 |
| HS7 | 3.09 | 8.79 | 13.11 |
| HS8 | 4.97 | 8.30 | 14.92 |
| Mean | 2.51 | 11.05 | 11.75 |

The inter- and intra-assay variation was a mean 11.75 and 11.05% respectively. For each assay a master calibrator prepared from synthetic peptides accurately quantified by amino acid analysis was used for calibration purposes. The analyte stability was determined for six serum samples (three rat and three human) for 10 freeze and thaw cycles. The analyte stability was found to be acceptable for 4 consecutive freeze/thaw cycles within 100+/−20%.

### Example 3

### IC6 ELISA vs Muscle Volume Assessments

Serum samples from 10 men (Table 3) were collected and the concentration of collagen type VI alpha 1 epitope determined using the herein described competition IC6 ELISA.

TABLE 3
subject demographics
Subject demographics

| | Young |
|---|---|
| n | 10 |
| Age | 24.4 ± 0.5 |
| Height | 181.4 ± 1.8 |
| Weight | 72.2 ± 2.3 |
| BMI | 22.1 ± 0.5 |
| Bodyfat | 24.5 + 5.7 |
| Activity level | 5.0 ± 0.9 |

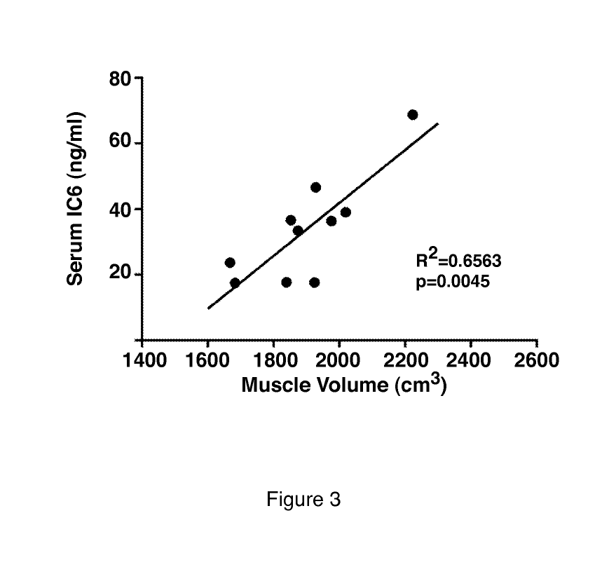

The muscle volume of each of the same 10 men was determined using MRI and these results were plotted against the above determined concentrations of collagen type VI alpha 1 epitope as assessed by the IC6 ELISA (FIG. 3). The concentration of collagen type VI alpha 1 epitope showed statistically significant correlation with muscle volume (R²=0.6523, p=0.0045).

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used in the sense of ‘including’ rather than in to mean ‘consisting of’. All prior teachings acknowledged above are hereby incorporated by reference. No acknowledgement of any prior published document herein should be taken to be an admission or representation that the teaching thereof was common general knowledge in Australia or elsewhere at the date hereof.

### REFERENCES

[1] Bosman, F. T., and Stamenkovic, I. Functional structure and composition of the extracellular matrix. J. Pathol. 2003;200:423-428.
[2] Bruckner, P. Suprastructures of extracellular matrices: paradigms of functions controlled by aggregates rather than molecules. Cell Tissue Res. 2010;339:7-18.
[3] D. Weil, M.-G. Mattei, E. Passage, N. Van Cong, D. Pribula-Conway, K. Mann, R. Deutzmann, R. Timpl, M.-L. Chu, Cloning and chromosomal localization of human genes encoding the three chains of type VI collagen, Am. J. Hum. Genet. 1988;42:435-445.
[4] M.-L. Chu, K.-H. Mann, R. Deutzmann, D. Pribula-Conway, C. C. Hsu-Chen, M. P. Bernard, R. Timpl, Characterization of three constituent chains of collagen type VI by peptide sequences and cDNA clones, Eur. J. Biochem. 1987;168:309-317.
[5] H. von der Mark, M. Aumailley, G. Wick, R. Fleischmajer, R. Timpl, Immunochemistry, genuine size and tissue localization of collagen VI, Eur. J. Biochem. 1984;142:493-502.
[6] R. Timpl, M.-L. Chu, Microfibrillar collagen type VI, in: P. D. Yuchenco, D. Birk, R. P. Mecham (Eds.), Extracellular Matrix Assembly and Structure, Academic Press, Orlando, 1994; pp. 207-242.
[7] D. R. Keene, E. Engvall, R. Glanville, Ultrastructure of type VI collagen in human skin and cartilage suggests an anchoring function for this filamenteous network, J. Cell Biol. 1988;107:1995-2006.
[8] J. Engel, H. Furthmayr, E. Obermatt, H. von der Mark, M. Aumailley, R. Fleischmajer, R. Timpl, Structure and macromolecular organization of type VI collagen, Ann. N.Y. Acad Sci. 1985;460:25-37.
[9] C. A. Poole, S. Ayad, R. T. Gilbert, Chondrons from articular cartilage-V. Immunohistochemical evaluation of type VI collagen organisation in isolated chondrons by light, confocal and electron microscopy, J. Cell. Sci. 1992;103:1101-1110.
[10] R. R. Bruns, Beaded filaments and long-spacing fibrils: relation to type VI collagen, J. Ultrastruct. Res. 1984;89:136-146.
[11] R. R. Bruns, W. Press, E. Engvall, R. Timpl, J. Gross, Type VI collagen in extracellular, 100-nm periodic filaments and fibrils: identification by immunoelecton microscopy, J. Cell Biol. 1986;103:393-404.
[12] M.-C. Ronzie're, S. Ricard-blum, J. Tiollier, D. J. Hartmann, R. Garrone, D. Herbage, Comparative analysis of collagens solubilized from human foetal, and normal and osteoarthritic adult articular cartilage, with emphasis on type VI collagen, Biochim. Biophys. Acta 1990;1038:222-230.
[13] Bonnemann C G. The collagen VI-related myopathies: muscle meets its matrix. Nat Rev Neurol 2011;7:379-90.
[14] Veidal S S, Karsdal M A, Vassiliadis E, Nawrocki A, Larsen M R, Nguyen Q H T, et al. MMP mediated degradation of type VI collagen is highly associated with liver fibrosis—identification and validation of a novel biochemical marker assay. PLoS ONE 2011;6:e24753.
[15] Gefter M L, Margulies D H, Scharff M D. A simple method for polyethylene glycol-promoted hybridization of mouse myeloma cells. Somatic Cell Genet. 1977;3:231-6.

1: An antibody, wherein said antibody binds to an epitope of collagen type VI alpha 1, said epitope being comprised in the N-terminal globular domain internal amino acid sequence -ADWGQSRDAEEAISQ- (SEQ ID NO: 1).

2: The [[An]] antibody as claimed in claim 1, wherein said antibody is a monoclonal antibody, or a polyclonal antibody, or an antibody fragment.

3: The [[An]] antibody as claimed in claim 1, wherein said antibody binds to an epitope comprised in the N-terminal globular domain internal amino acid sequence -ADWGQSRDAEEAISQ- (SEQ ID NO: 1) of human or rodent collagen type VI alpha 1.

4: A method of immunoassay for detecting in a biological sample an epitope comprised in the N-terminal globular domain internal amino acid sequence -ADWGQSRDAEEAISQ- (SEQ ID NO: 1) of collagen type VI alpha 1, said method comprising contacting said biological sample comprising said epitope comprised in said N-terminal globular domain internal amino acid sequence -ADWGQSRDAEEAISQ- (SEQ ID NO: 1) of collagen type VI alpha 1 with an antibody as claimed in claim 1, and determining the amount of binding of said antibody.

5: The method as claimed in claim 4, wherein said method is used to quantify the amount of intact collagen type VI alpha 1 and fragments thereof comprising the amino acid sequence ADWGQSRDAEEAISQ (SEQ ID NO: 1) in biofluid.

6: The method as claimed in claim 5, wherein said biofluid is serum or plasma.

7: The method as claimed in claim 4, wherein said immunoassay is a competition assay or a sandwich assay.

8: The method as claimed in claim 4, wherein said immunoassay is a radioimmunoassay or an enzyme-linked immunosorbent assay.

9: The method as claimed in claim 5, further comprising correlating the quantity of intact collagen type VI alpha 1 and fragments thereof comprising said amino acid sequence ADWGQSRDAEEAISQ (SEQ ID NO: 1) determined by said method with standard disease samples of known disease severity to evaluate the severity of a disease associated with collagen type VI alpha 1.

10: The method as claimed in claim 5, further comprising correlating the quantity of intact collagen type VI alpha 1 and fragments thereof comprising said amino acid sequence ADWGQSRDAEEAISQ (SEQ ID NO: 1) determined by said method with MRI-determined muscle volume.

11: An assay kit for determining the quantity of intact collagen type VI alpha 1 and fragments thereof comprising the amino acid sequence ADWGQSRDAEEAISQ (SEQ ID NO: 1) in a biological sample, said kit comprising an antibody as claimed in claims 1 and at least one of:
a streptavidin coated 96 well plate;
a biotinylated peptide Biotin-L-ADWGQSRDAEEAISQ (SEQ ID NO: 2), wherein L is an optional linker;
a secondary antibody for use in a sandwich immunoassay;
a calibrator peptide comprising the sequence ADWGQSRDAEEAISQ (SEQ ID NO: 1) or a portion thereof which binds to said antibody;
an antibody biotinylation kit;
an antibody HRP labeling kit;
an antibody radiolabeling kit; or
an assay visualization kit.
